(19) United States
(12) Patent Application Publication
(10) Pub. No.: US 2023/0075422 A1
(43) Pub. Date: Mar. 9, 2023

(54) USE OF FUCOSYLATION INHIBITOR FOR PRODUCING AFUCOSYLATED ANTIBODY

(71) Applicant: BRISTOL-MYERS SQUIBB COMPANY, Princeton (US)

(72) Inventors: Ping XU, Doylestown, NJ (US); Anurag Khetan, Princeton, NJ (US); Rodney Lawrence Parsons, Yardley, PA (US); Michael J. Smith, Somerset, NJ (US); Michael Anthony Schmidt, Cranbury, NJ (US)

(21) Appl. No.: 17/787,461

(22) PCT Filed: Dec. 18, 2020
(86) PCT No.: PCT/US2020/065982

§ 371 Date: Jun. 20, 2022

Related U.S. Application Data
(60) Provisional application No. 62/951,318, filed on Dec. 20, 2019.

(51) Int. Cl. C07K 16/00; C12N 5/07; A61P 35/00

(52) U.S. Cl. CPC C07K 16/00; C12N 5/06; A61P 35/00; C12N 2500/34; C07K 2317/732; C07K 2317/41

(57) ABSTRACT

The present invention provides inhibitors of fucosylation during protein expression from mammalian cells. The inhibitors are derived from rhamnose and act by inhibition of GDP-mannose 4,6-dehydratase (GMD). The invention further provides methods of making proteins with reduced level of fucosylation, such as antibodies and antibodies made by the methods of the present invention. Such hypofucosylated or nonfucosylated antibodies may find use, for example, in treatment of human disease in which is it therapeutically eneficial to direct antibody dependent cellular cytotoxicity (ADCC) mediated killing of cells expressing the antibody target on their surface, for example in depletion of Tregs in cancer patients using a hypofucosylated or nonfucosylated anti-CTLA-4 antibody.

### CROSS REFERENCE TO RELATED APPLICATIONS

This application claims priority to U.S. Provisional Application No. 62/951,318, filed 20 Dec. 2019, the disclosure of which is incorporated herein by reference.

### SEQUENCE LISTING

The Sequence Listing filed electronically herewith is also hereby incorporated by reference in its entirety (File Name: 202001104_SEQL_13347WOPCT_GB.txt; Date Created: 4 Nov. 2020; File Size: 11 KB).

### BACKGROUND OF THE INVENTION

Therapeutic antibodies are more and more commonly used to treat human disease. Antibodies are generated that bind to targets of therapeutic interest, and are selected and modified to exhibit a desired effect on a disease mechanism. Treatment of autoimmune diseases has been revolutionized through use of antibodies that bind to inflammatory mediators, such as cytokines and their receptors. Such antibodies typically are intended simply to block an inflammatory signaling pathway, and need do little more than bind to a target protein at an epitope that blocks binding to its ligand or receptor.

Antibodies have also been developed for the treatment of cancer. The original therapeutic model of anti-cancer antibodies was the idea of the “magic bullet” that directs toxic drugs specifically to tumor cells. Antibodies would be raised to tumor-specific cell surface antigens, and then derivatized with a cytotoxic “payload,” often a conventional chemotherapy agent. When administered to a cancer patient the antibody would circulate and bind specifically to tumor cells, delivering the toxic payload only to tumor cells and largely sparing healthy tissue, thus reducing side effects. A drug could be attached by a linker that would release the cytotoxin in the vicinity of the target tumor cell, creating a locally high concentration at the tumor, or it could remain attached to the antibody until the antibody was internalized after binding to a cell surface receptor.

An alternative to a cytotoxic payload is use of antibodies capable of directing an enhanced immune response specifically to tumor cells. As in the magic bullet approach, antibodies direct cytotoxicity to tumor cells, but in this case they direct cytotoxic immune response. Such antibodies must be designed not only to bind to tumor-specific cell surface markers, but also to attract and/or activate immune cells, such as anti-tumor CD8⁺ T cells, to the vicinity of the tumor.

An even more recent approach to treatment of cancer with antibodies is immuno-oncology. In this approach, antibodies are designed not to kill tumor cells directly, but instead by modifying the activity of the immune system to elicit an effective anti-tumor immune response. It has been found that many tumors elicit an anti-tumor immune response, but that this immune response is thwarted by the activity of various cell surface receptors that block signals that activate anti-tumor response, or that enhance immunosuppressive mechanisms. Immunosuppressive mechanisms are essential to restore homeostasis, and otherwise limit immune responses after they are no longer needed, but these mechanisms may inhibit anti-tumor immune responses when such responses would be beneficial. One such immunosuppressive factor is regulatory T cells (Tregs), which are a subset of T cells that function to suppress the activity of cytotoxic CD8+ T cells. In a patient with a life-threatening tumor, such suppressive effects may permit growth of a tumor that might otherwise be eliminated or controlled. In fact, the presence of high levels of Tregs within a tumor is a known marker for poor prognosis. Tao et al. (2012) Lung Cancer 75:95.

As a consequence, it is beneficial in treatment of some cancers to deplete the population of Tregs so as to allow an unfettered anti-tumor immune response. As with tumor cells, one approach is to use antibodies specific for Tregs, such as anti-CTLA-4 or anti-CCR4. Such antibodies are designed to deplete Tregs and may do so by directing an immune response against those cells, for example by antibody-dependent cellular cytotoxicity (ADCC) effected by CD8+T cells. Antibodies are designed with Fc regions that bind to activating Fc receptors on T cells to increase anti-tumor immune response—such antibodies are said to have effector function. Effector function may be enhanced by modification of the Fc portion of the antibody that interacts with immune cells, such as by modifying the amino acid sequence of the Fc region or modifying the glycosylation.

It has also been found that elimination of fucose from N-linked glycan chains at N297 of human immunoglobulin heavy chains leads to enhanced binding to activating Fc receptors, resulting in greatly enhanced anti-tumor ADCC-mediated toxicity. Rothman et al. (1989) Mol. Immunol. 26:1113, at 1122 (proposing reduction in core fucosylation of antibodies to enhance ADCC of antibodies used in immunotherapy of neoplasias); Harris et al. (1997) Biochemistry 36:1581; Satoh et al. (2003) Expert Opin. Bio. Ther. 6:1161.

Several methods are known to generate such antibodies with reduced fucosylation, including hypofucosylated and nonfucosylated antibodies. Le et al. (2016) Biochim. Biophys. Acta 1860:1655. Antibodies can be produced in cell lines that are naturally deficient in fucosylation (Lifely et al. (1995) Glycobiology 5:813), or in cell lines in which key enzymatic components of the fucosylation pathway have been knocked out, for example in cells lacking Fucosyl Transferase 8 (FUT8) such as POTELLIGENT® Chinese hamster ovary (CHO) cells. See, e.g., Rothman et al. (1989) Mol. Immunol. 26:1113; WO 97/27303; WO 99/54342; WO 00/61739; WO 02/31140. Alternatively, inhibitors of the enzymatic fucosylation pathway may be added to cultures during production of antibodies. See, e.g., Rothman et al. (1989) Mol. Immunol. 26:1113; U.S. Pat. No. 8,071,336; WO 09/135181; WO 14/130613; EP 2958905 B1; Allen et al. (2016) ACS Chem. Biol. 11:2734. Exemplary small molecule inhibitors of fucosylation include, but are not limited to, castanospermine, 2F-peracetyl-fucose, 2-deoxy-2-fluoro-L-fucose, 6,6,6,-trifulorofucose (Fucostatin I) and 6,6,6,-trifulorofucose phosphonate analog (Fucostatin II). Rothman et al. (1989) Mol. Immunol. 26:1113; Okeley et al. (2016) Proc. Nat'l Acad. Sci. (USA) 110:5404; Rillahan et al. (2012) Nat. Chem. Biol. 8:661; U.S. Pat. No. 8,163,551; EP 2958905 B1; Allen et al. (2016) ACS Chem. Biol. 11:2734. Other creative approaches include enzymatic depletion of GDP-fucose precursors in antibody production cell lines, GLYMAXX® fucosylation inhibition technology. See, e.g., U.S. Pat. No. 8,642,292; von Horsten et al. (2010) Glycobiology 20:1607; Roy et al. (2018) mAbs 10:416.

The need exists for hypofucosylated and nonfucosylated antibodies, and improved methods of making them. A method that allows tunable increase and decrease in percentage of molecules with fucosylation could be particularly valuable in discovery research. Ideally, such methods would not require introduction of any genetic constructs into cell lines used for antibody production, or time consuming creation of new stable cell lines, and would not significantly reduce the titer of antibody produced compared with production of fucosylated antibody.

### SUMMARY OF THE INVENTION

The present invention provides compounds for use as fucosylation inhibitors that inhibit mammalian GDP-mannose 4,6-dehydratase (GMD), e.g. hamster GMD. Such compounds will find use, for example, in manufacture of proteins, such as antibodies, with reduced N-linked glycan fucosylation, in which said compounds are added to cell cultures during production of the protein (e.g. antibody).

In various embodiments the compound of the present invention is a derivative of rhamnose, such as GDP-D-rhamnose, Ac-GDP-D-rhamnose or sodium rhamnose phosphate. In one embodiment GDP-D-rhamnose is the compound of the present invention. In another embodiment, Ac-GDP-D-rhamnose is the compound of the present invention. In yet another embodiment, sodium rhamnose phosphate is the compound of the present invention. In various embodiments the fucosylation inhibitor of the present invention is present at 6 mM or higher concentration, or 10 mM or higher concentration, in the culture medium.

In another aspect, the invention provides methods of making proteins, such as antibodies, with reduced fucosylation by including the compounds of the present invention in the culture medium used during production of the proteins from a cell line expressing the proteins, e.g. antibodies. In some embodiments the compounds are present in the culture medium for all or substantially all of the time during which protein (e.g. antibody) to be isolated is being produced by the cell line, to maximize the proportion of nonfucosylated protein (e.g. antibody) produced, although in principal the compound need only be present during enough of the production culture to attain the desired level of nonfucosylation.

In a further aspect, the invention provides proteins with reduced fucosylation made by the methods of the present invention, such as proteins with reduced fucosylation (e.g. ≥20% or ≥40% afucosylated polypeptide chains), or hypofucosylated or nonfucosylated proteins.

In a related aspect, the invention provides antibodies with reduced fucosylation made by the methods of the present invention, such as antibodies that exhibit two-fold or greater enhancement of ADCC compared with the same antibody produced in the same cell line in the absence of fucosylation inhibitor (as determined by the method described in Example 2), and/or antibodies with reduced fucosylation (e.g. ≥20% or ≥40% afucosylated antibody chains), or hypofucosylated or nonfucosylated antibodies.

In an even further aspect, the invention provides methods of treatment of human diseases such as cancer, by administering antibodies or other proteins with reduced fucosylation made by the methods of the present invention to patients in need thereof.

In various embodiments, the compound of the present invention is included in the cell growth medium used during antibody production at a concentration of 1 mM, 2 mM, 3 mM, 6 mM, 10 mM or higher.

Exemplary antibodies that can be made in hypofucosylated or nonfucosylated form by the methods of the present invention include antibodies binding to human CD20, CCR4, EGFR, CD19, Her2, IL-5R, CD40, BCMA, Siglec 8, CD147, CD30, EphA3, Fucosyl GM1, CTLA-4, MICA, and ICOS.

### BRIEF DESCRIPTION OF THE DRAWINGS

FIG. 1 provides structures for three compounds, specifically GDP-D-rhamnose (Formula I), Ac-GDP-D-rhamnose (Formula II), and sodium rhamnose phosphate (Formula IIII).

FIG. 2A shows the percentage of nonfucosylated antibodies for antibodies grown in the presence or absence of exemplary fucosylation inhibitors of the present invention at concentrations from 1 mM to 6 mM. The structures of GDP-D-rhamnose and Ac-GDP-D-rhamnose are provided in FIG. 1. FIG. 2B shows the antibody titers for the same antibody preparations of FIG. 2A. Ac-GDP-D-rhamnose is as effective as GDP-D-rhamnose in increasing the percentage of nonfucosylated antibodies with a less deleterious effect on titer (yield).

FIGS. 3A, 3B and 3C show electropherograms of antibody preparations made using cells cultured without a fucosylation inhibitor or in the presence of 6 mM or 10 mM Ac-GDP-D-rhamnose, respectively. Peaks for different glycoforms are indicated, with white boxes for fucosylated species and dark boxes for nonfucosylated species. Increasing concentrations of Ac-GDP-D-rhamnose increase the proportion of nonfucosylated species.

FIGS. 4A and 4B provide an exemplary synthetic scheme for the production of rhamnose phosphate, GDP-D-rhamnose and Ac-GDP-D-rhamnose. See Example 1.

FIGS. 5A, 5B and 5C provide a second exemplary synthetic scheme for the production of GDP-D-rhamnose and Ac-GDP-D-rhamnose.

### DETAILED DESCRIPTION OF THE INVENTION

### Definitions

In order that the present disclosure may be more readily understood, certain terms are first defined. As used in this application, except as otherwise expressly provided herein, each of the following terms shall have the meaning set forth below. Additional definitions are set forth throughout the application.

“Administering” refers to the physical introduction of a composition comprising a therapeutic agent to a subject, using any of the various methods and delivery systems known to those skilled in the art. Preferred routes of administration for antibodies of the invention include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody of the invention can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods. Administration may be performed by one or more individual, including but not limited to, a doctor, a nurse, another healthcare provider, or the patient himself or herself “A patient in need thereof” as recited in the claims, refers to any human subject diagnosed with the disease to be treated, such as cancer.

An “antibody” (Ab) shall include, without limitation, a glycoprotein immunoglobulin which binds specifically to an antigen and comprises at least two heavy (H) chains and two light (L) chains interconnected by disulfide bonds. Antibodies made by the methods of the present invention, which include production of an antibody in cell lines cultured in the presence of a fucosylation inhibitor of the present invention, are referred to as antibodies of the present invention. In a conventional antibody, each H chain comprises a heavy chain variable region (abbreviated herein as V_{H}) and a heavy chain constant region. The heavy chain constant region comprises three domains, C_{H1}, C_{H2} and C_{H3}. Each light chain comprises a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen.

As used herein, and in accord with conventional interpretation, an antibody that is described as comprising “a” heavy chain and/or “a” light chain refers to antibodies that comprise “at least one” of the recited heavy and/or light chains, and thus will encompass antibodies having two or more heavy and/or light chains. Specifically, antibodies so described will encompass conventional antibodies having two substantially identical heavy chains and two substantially identical light chains. Antibody chains may be substantially identical but not entirely identical if they differ due to post-translational modifications, such as C-terminal cleavage of lysine residues, alternative glycosylation patterns, etc. An “antibody” may also comprise two distinct antigen binding domains, e.g. a bispecific antibody or an antibody binding to two different epitopes on the same target, and thus may comprise two non-identical heavy and/or light chains.

Unless indicated otherwise or clear from the context, an antibody defined by its target specificity (e.g. an “anti-CTLA-4 antibody”) refers to antibodies that can bind to its human target (e.g. human CTLA-4). Such antibodies may or may not bind to CTLA-4 from other species.

The immunoglobulin may derive from any of the commonly known isotypes, including but not limited to IgA, secretory IgA, IgG and IgM. The IgG isotype may be divided in subclasses in certain species: IgG1, IgG2, IgG3 and IgG4 in humans, and IgG1, IgG2a, IgG2b and IgG3 in mice. IgG antibodies may be referred to herein by the symbol gamma (γ) or simply “G,” e.g. IgG1 may be expressed as “γ1” or as “G1,” as will be clear from the context. “Isotype” refers to the antibody class (e.g., IgM or IgG1) that is encoded by the heavy chain constant region genes. “Antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. Unless otherwise indicated, or clear from the context, antibodies disclosed herein are human IgG1 antibodies.

An “isolated antibody” refers to an antibody that is substantially free of other antibodies having different antigenic specificities (e.g., an isolated antibody that binds specifically to CTLA-4 is substantially free of antibodies that bind specifically to antigens other than CTLA-4). An isolated antibody that binds specifically to CTLA-4 may, however, cross-react with other antigens, such as CTLA-4 molecules from different species. Moreover, an isolated antibody may be substantially free of other cellular material and/or chemicals. By comparison, an “isolated” nucleic acid refers to a nucleic acid composition of matter that is markedly different, i.e., has a distinctive chemical identity, nature and utility, from nucleic acids as they exist in nature. For example, an isolated DNA, unlike native DNA, is a free-standing portion of a native DNA and not an integral part of a larger structural complex, the chromosome, found in nature. Further, an isolated DNA, unlike native DNA, can be used as a PCR primer or a hybridization probe for, among other things, measuring gene expression and detecting biomarker genes or mutations for diagnosing disease or predicting the efficacy of a therapeutic. An isolated nucleic acid may also be purified so as to be substantially free of other cellular components or other contaminants, e.g., other cellular nucleic acids or proteins, using standard techniques well known in the art.

The term “monoclonal antibody” (“mAb”) refers to a preparation of antibody molecules of single molecular composition, i.e., antibody molecules whose primary sequences are essentially identical, and which exhibits a single binding specificity and affinity for a particular epitope. Monoclonal antibodies may be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. The terms “human” antibodies and “fully human” antibodies and are used synonymously.

An “antibody fragment” refers to a portion of a whole antibody, generally including the “antigen-binding portion” (“antigen-binding fragment”) of an intact antibody which retains the ability to bind specifically to the antigen bound by the intact antibody, or the Fc region of an antibody which retains FcR binding capability. Exemplary antibody fragments include Fab fragments and single chain variable domain (scFv) fragments.

“Antibody-dependent cell-mediated cytotoxicity” (“ADCC”) refers to an in vitro or in vivo cell-mediated reaction in which nonspecific cytotoxic cells that express FcRs (e.g., natural killer (NK) cells, macrophages, neutrophils and eosinophils) recognize antibody bound to a surface antigen on a target cell and subsequently cause lysis of the target cell. In principle, any effector cell with an activating FcR can be triggered to mediate ADCC.

“Cancer” refers a broad group of various diseases characterized by the uncontrolled growth of abnormal cells in the body. Unregulated cell division and growth divide and grow results in the formation of malignant tumors or cells that invade neighboring tissues and may also metastasize to distant parts of the body through the lymphatic system or bloodstream.

A “cell surface receptor” refers to molecules and complexes of molecules capable of receiving a signal and transmitting such a signal across the plasma membrane of a cell.

An “effector cell” refers to a cell of the immune system that expresses one or more FcRs and mediates one or more effector functions. Preferably, the cell expresses at least one type of an activating Fc receptor, such as, for example, human FcγRIII, and performs ADCC effector function. Examples of human leukocytes which mediate ADCC include peripheral blood mononuclear cells (PBMCs), NK cells, monocytes, macrophages, neutrophils and eosinophils.

“Effector function” refers to the interaction of an antibody Fc region with an Fc receptor or ligand, or a biochemical event that results therefrom. Exemplary “effector functions” include Clq binding, complement dependent cytotoxicity (CDC), Fc receptor binding, FcγR-mediated effector functions such as ADCC and antibody dependent cell-mediated phagocytosis (ADCP), and down-regulation of a cell surface receptor (e.g., the B cell receptor; BCR). Such effector functions generally require the Fc region to be combined with a binding domain (e.g., an antibody variable domain).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) receptors and one inhibitory (FcγRIIB) receptor. Various properties of human FcγRs are summarized in Table 1. The majority of innate effector cell types coexpress one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, the Fc region is a polypeptide comprising the constant region of an antibody excluding the first constant region immunoglobulin domain. In IgG, IgA and IgD antibody isotypes, the Fc region is composed of two identical protein fragments, derived from the second (C_{H2}) and third (C_{H2}) constant domains of the antibody's two heavy chains; IgM and IgE Fc regions contain three heavy chain constant domains (C_{H} domains 2-4) in each polypeptide chain. For IgG, the Fc region comprises immunoglobulin domains Cγ2 and Cγ3 and the hinge between Cγ1 and Cγ2. Although the boundaries of the Fc region of an immunoglobulin heavy chain might vary, the human IgG heavy chain Fc region is usually defined to stretch from an amino acid residue at position C226 or P230 to the carboxy-terminus of the heavy chain, wherein the numbering is according to the EU index as in Kabat. The C_{H2} domain of a human IgG Fc region extends from about amino acid 231 to about amino acid 340, whereas the C_{H3} domain is positioned on C-terminal side of a C_{H2} domain in an Fc region, i.e., it extends from about amino acid 341 to about amino acid 447 of an IgG. As used herein, the Fc region may be a native sequence Fc or a variant Fc. Fc may also refer to this region in isolation or in the context of an Fc-comprising protein polypeptide such as a “binding protein comprising an Fc region,” also referred to as an “Fc fusion protein” (e.g., an antibody or immunoadhesin).

TABLE 1
Properties of Human FcγRs

| | | Affinity | | |
|---|---|---|---|---|
| | Allelic | for human | Isotype | |
| Fcγ | variants | IgG | preference | Cellular distribution |
| FcγRI | None | High | IgG1 = 3 > | Monocytes, |
| | described | (K_{D} ~10 | 4 >> 2 | macrophages, |
| | | nM) | | activated neutrophils, |
| | | | | dendritic cells? |
| FcγRIIA | H131 | Low to | IgG1 > 3 > | Neutrophils, |
| | | medium | 2 > 4 | monocytes, |
| | R131 | Low | IgG1 > 3 > | macrophages, |
| | | | 4 > 2 | eosinophils, dendritic |
| | | | | cells, platelets |
| FcγRIIIA | V158 | Medium | IgG1 = 3 >> | NK cells, monocytes, |
| | | | 4 > 2 | macrophages, mast |
| | F158 | Low | IgG1 = 3 >> | cells, eosinophils, |
| | | | 4 > 2 | dendritic cells? |
| FcγRIIB | I232 | Low | IgG1 = 3 = | B cells, monocytes, |
| | | | 4 > 2 | macrophages, dendritic |
| | T232 | Low | IgG1 = 3 = | cells, mast cells |
| | | | 4 > 2 | |

“Fucosylation,” as used herein unless otherwise indicated, refers to the presence of a branched fucose residue at the innermost GlcNac residue of an N-linked glycan chain on a protein. Fucosylation is a bulk property of a population of protein molecules, although the term may also be used with reference to individual proteins within the population. Any individual antibody, for example, may be “fucosylated” on both heavy chains (fucosylated), on neither heavy chain (nonfucoslyated), or on only one of the two heavy chains (hemi-fucosylated). A population of antibodies, for example a preparation from a production run, will comprise a mixture of individual fucosylated, nonfucosylated and hemi-fucosylated antibodies and thus may exhibit any degree of fucosylation from 0% to 100%. Percent fucosylation, as used herein, refers to the percentage of all potential fucosylation sites having a fucose present. For example, a preparation of pure hemi-fucosylated antibodies would be 50% fucosylated. Exemplary methods of determining the percent fucosylation in a preparation of antibodies are provided at Example 2.

GMD refers to “GDP-mannose 4,6-dehydratase” from a mammal, such as hamster or human. GMD is referred to by Enzyme Commission (EC) number 4.2.1.47. Human GMD is also referred to as GMDS and SDR3E1. GMD catalyzes the conversion of GDP-mannose to GDP-4-keto-6-deoxymannose, the first step in the synthesis of GDP-fucose from GDP-mannose, using NADP+ as a cofactor. Unless otherwise indicated, or clear from the context, references to GMD herein refer to hamster GMD, although it most contexts both hamster and human proteins will be included. Hamster (Cricetulus griseus) GMD is further described at GENE ID NO: 100689436. The sequence of hamster GMD (NP_001233625.1), including 23 amino acid signal sequence, is provided at SEQ ID NO: 1, with the encoding DNA sequence NM_001246696.1 provided at SEQ ID NO: 2. Human (Homo sapiens) GMD is further described at GENE ID NO: 2762 and MIM (Mendelian Inheritance in Man): 602884. The sequence of human GMD isoform 1 (NP_001491.1), including 23 amino acid signal sequence, is provided at SEQ ID NO: 3, with the encoding DNA sequence NM_001500.4 provided at SEQ ID NO: 4. The hamster and human GMD polypeptides share 98% sequence similarity and >99% sequence identity over the 347aa mature protein.

An “immune response” refers to a biological response within a vertebrate against foreign agents, which response protects the organism against these agents and diseases caused by them. The immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues.

An “immunomodulator” or “immunoregulator” refers to a component of a signaling pathway that may be involved in modulating, regulating, or modifying an immune response. “Modulating,” “regulating,” or “modifying” an immune response refers to any alteration in a cell of the immune system or in the activity of such cell. Such modulation includes stimulation or suppression of the immune system which may be manifested by an increase or decrease in the number of various cell types, an increase or decrease in the activity of these cells, or any other changes which can occur within the immune system. Both inhibitory and stimulatory immunomodulators have been identified, some of which may have enhanced function in a tumor microenvironment. In preferred embodiments of the disclosed invention, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying an immune response.

“Potentiating an endogenous immune response” means increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response.

A “protein” refers to a chain comprising at least two consecutively linked amino acid residues, with no upper limit on the length of the chain. One or more amino acid residues in the protein may contain a modification such as, but not limited to, glycosylation, phosphorylation or disulfide bond formation. The term “protein” is used interchangeable herein with “polypeptide.” A “protein” may comprise two or more polypeptide chains, which may comprise different polypeptide sequences, such as the heavy and light chains of an antibody. A conventional full-length antibody will comprise two heavy chains and two light chains, and is a “protein.” A cell or cell line expressing a “protein” comprising two or more polypeptides having different sequences expresses all of the chains of the protein, for example both the heavy and light chains of an antibody.

A “protein,” as used herein, unless otherwise indicated, and with reference to the compounds and method of the present invention to produce proteins with reduced fucosylation, comprises an N-linked glycan. Proteins with N-linked glycosylation, such as Fc region (N297) glycosylation in antibodies, may be used with the compounds and methods of the present invention to limit or prevent addition of the fucose residue otherwise typically added to the innermost GlcNac residue of the glycan chain.

As is conventional, the term “protein,” such as an “antibody,” may refer to either a population of protein molecules in a preparation, or an individual protein molecule within that population, depending on the context. For clarity, the term “afucosylated” is used herein to refer to individual protein (e.g. antibody chain) lacking N-linked fucose, and “nonfucosylated” is used to refer to populations or preparations of protein molecules. As a consequence, any individual polypeptide chain may be either fucosylated or afucosylated, whereas a population of proteins may be nonfucosylated to any given percentage of afucosylation. Accordingly, any reference to a protein or proteins with reference to a level of fucosylation, e.g. “an antibody with reduced fucosylation,” necessarily refers to a heterogeneous population of protein molecules even when not expressly stated.

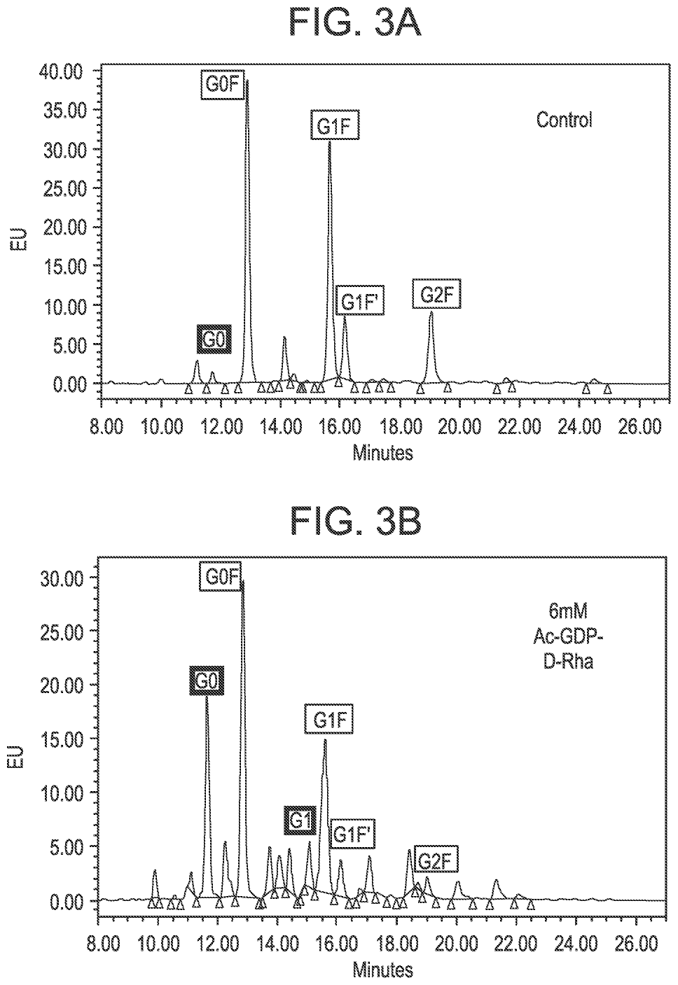

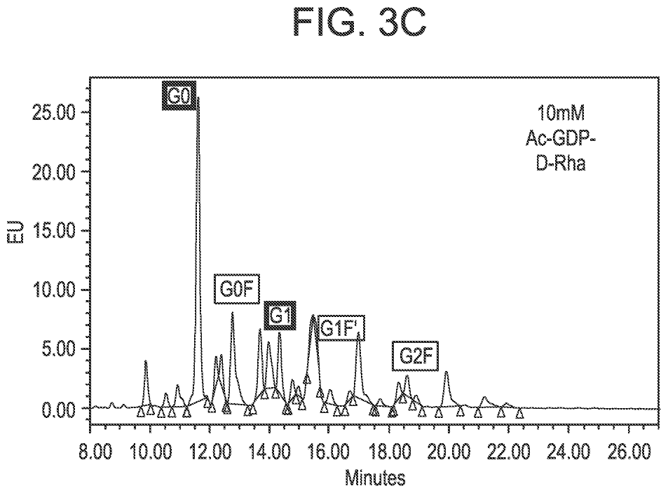

Unless otherwise indicated, or clear from the context, amino acid residue numbering in the Fc region of an antibody is according to the EU numbering convention (the EU index as in Kabat et al. (1991) Sequences of Proteins of Immunological Interest, National Institutes of Health, Bethesda, Md.; see also FIGS. 3c-3f of U.S. Pat. App. Pub. No. 2008/0248028), except when specifically referring to residues in a sequence in the Sequence Listing, in which case numbering is necessarily consecutive. For example, literature references regarding the effects of amino acid substitutions in the Fc region will typically use EU numbering, which allows for reference to any given residue in the Fc region of an antibody by the same number regardless of the length of the variable domain to which is it attached. In rare cases it may be necessary to refer to the document being referenced to confirm the precise Fc residue being referred to.

“Rhamnose,” unless otherwise indicated, refers to D-rhamnose.

A “subject” includes any human or non-human animal. The term “non-human animal” includes, but is not limited to, vertebrates such as nonhuman primates, sheep, dogs, rabbits, rodents such as mice, rats and guinea pigs, avian species such as chickens, amphibians, and reptiles. In preferred embodiments, the subject is a mammal such as a nonhuman primate, sheep, dog, cat, rabbit, ferret or rodent. In more preferred embodiments of any aspect of the disclosed invention, the subject is a human. The terms, “subject” and “patient” are used interchangeably herein.

“Treatment” or “therapy” of a subject refers to any type of intervention or process performed on, or administering an active agent to, the subject with the objective of reversing, alleviating, ameliorating, inhibiting, slowing down or prevent the onset, progression, development, severity or recurrence of a symptom, complication, condition or biochemical indicia associated with a disease.

### Traditional Methods of Reducing Fucosylation of Antibodies

The interaction of antibodies with FcγRs can be enhanced by modifying the glycan moiety attached to each Fc fragment at the N297 residue. In particular, the absence of core fucose residues strongly enhances ADCC via improved binding of IgG to activating FcγRIIIA without altering antigen binding or CDC. Natsume et al. (2009) Drug Des. Devel. Ther. 3:7. There is convincing evidence that afucosylated tumor-specific antibodies translate into enhanced therapeutic activity in mouse models in vivo. Nimmerjahn & Ravetch (2005) Science 310:1510; Mossner et al. (2010) Blood 115:4393.

Modification of antibody glycosylation has traditionally been accomplished by, for example, expressing the antibody in a host cell with altered glycosylation machinery. Cells with altered glycosylation machinery have been described in the art. For example, the cell lines Ms704, Ms705, and Ms709 lack the fucosyltransferase gene, FUT8 (α-(1,6) fucosyltransferase (see U.S. Pat. App. Publication No. 20040110704; Yamane-Ohnuki et al. (2004) Biotechnol. Bioeng. 87: 614), such that antibodies expressed in these cell lines lack fucose on their carbohydrates. EP 1176195 also describes a cell line with a functionally disrupted FUT8 gene as well as cell lines that have little or no activity for adding fucose to the N-acetylglucosamine that binds to the Fc region of the antibody, for example, the rat myeloma cell line YB2/0 (ATCC CRL 1662). PCT Publication WO 03/035835 describes a variant CHO cell line, Lec13, with reduced ability to attach fucose to Asn(297)-linked carbohydrates, also resulting in hypofucosylation of antibodies expressed in that host cell. See also Shields et al. (2002) J. Biol. Chem. 277:26733. Antibodies with a modified glycosylation profile can also be produced in chicken eggs, as described in PCT Publication No. WO 2006/089231. Alternatively, antibodies with a modified glycosylation profile can be produced in plant cells, such as Lemna. See e.g. U.S. Publication No. 2012/0276086. PCT Publication No. WO 99/54342 describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies. See also Umaña et al. (1999) Nat. Biotech. 17:176. Alternatively, the fucose residues of the antibody may be cleaved off using a fucosidase enzyme. For example, the enzyme alpha-L-fucosidase removes fucosyl residues from antibodies. Tarentino et al. (1975) Biochem. 14:5516. Antibodies with reduced fucosylation may also be produced in cells harboring a recombinant gene encoding an enzyme that uses GDP-6-deoxy-D-lyxo-4-hexylose as a substrate, such as GDP-6-deoxy-D-lyxo-4-hexylose reductase (RMD), as described at U.S. Pat. No. 8,642,292. Alternatively, cells may be grown in medium containing fucose analogs that block the addition of fucose residues to the N-linked glycan or a glycoprotein, such as antibody, produced by cells grown in the medium. U.S. Pat. No. 8,163,551; WO 09/135181. Such compounds include, but are not limited to, peracetyl-fucose, 6,6,6-trifulorofucose per-O-acetate, 6,6,6-trifulorofucose (Fucostatin I) and a fucose phosphate analog (Fucostatin II).

### Rhamnose Derivatives as Fucosylation Inhibitors

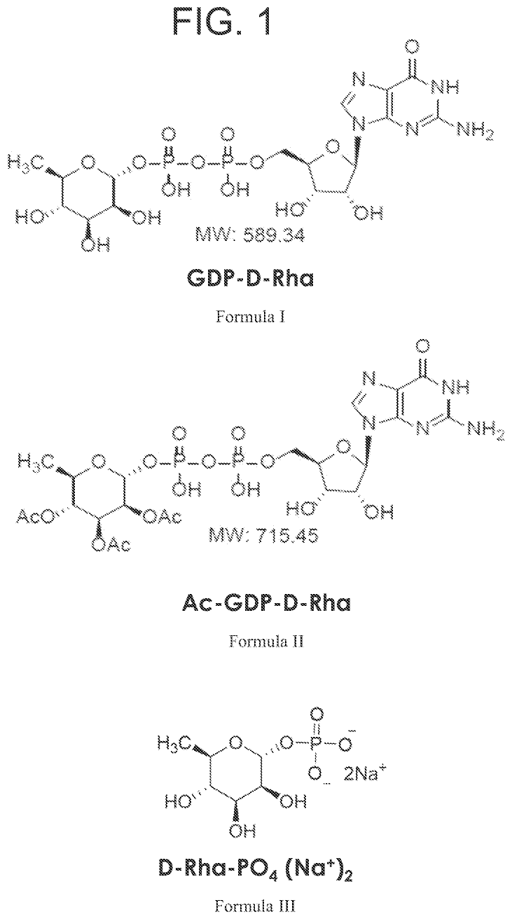

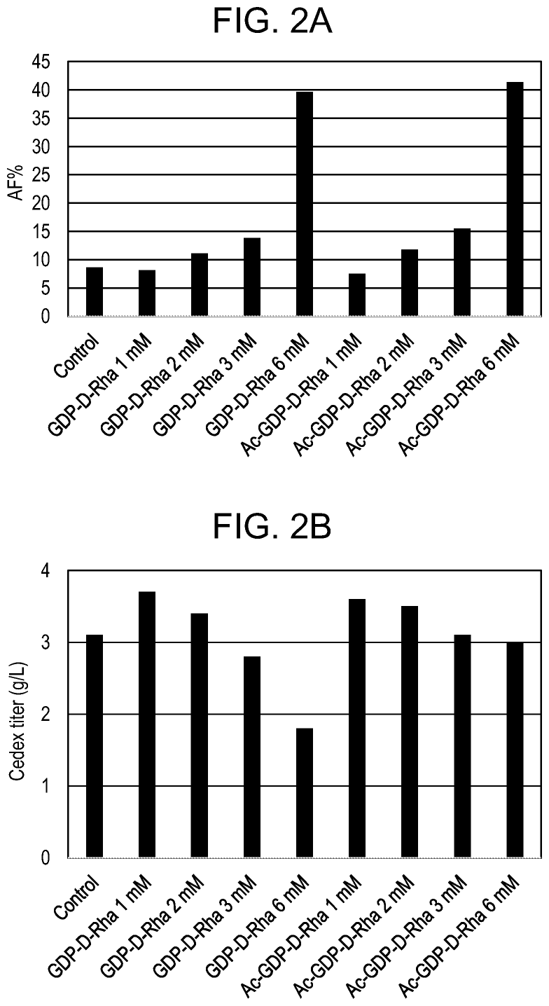

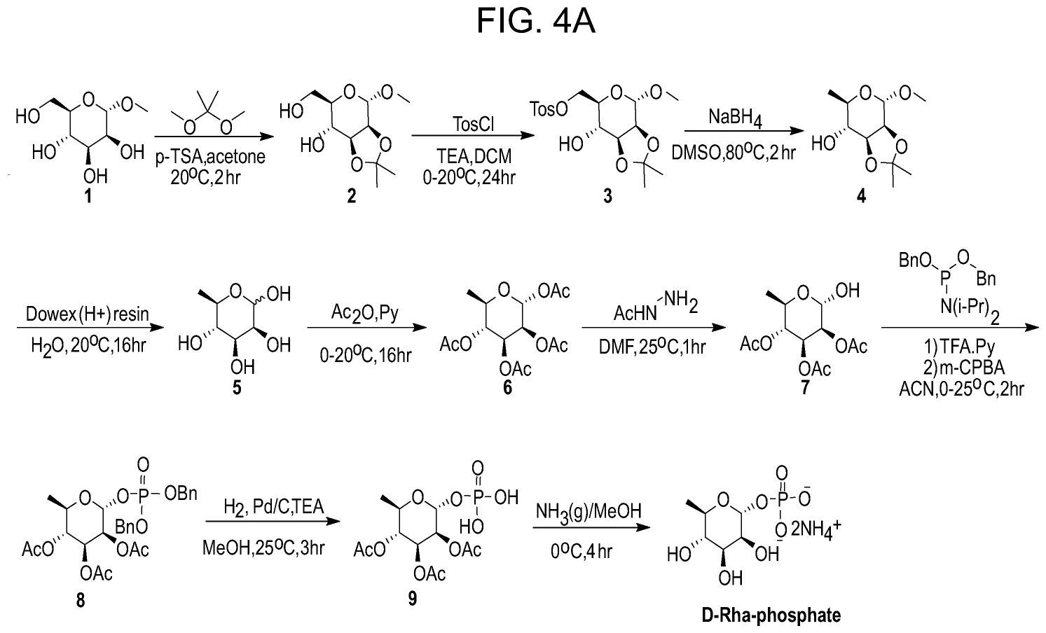

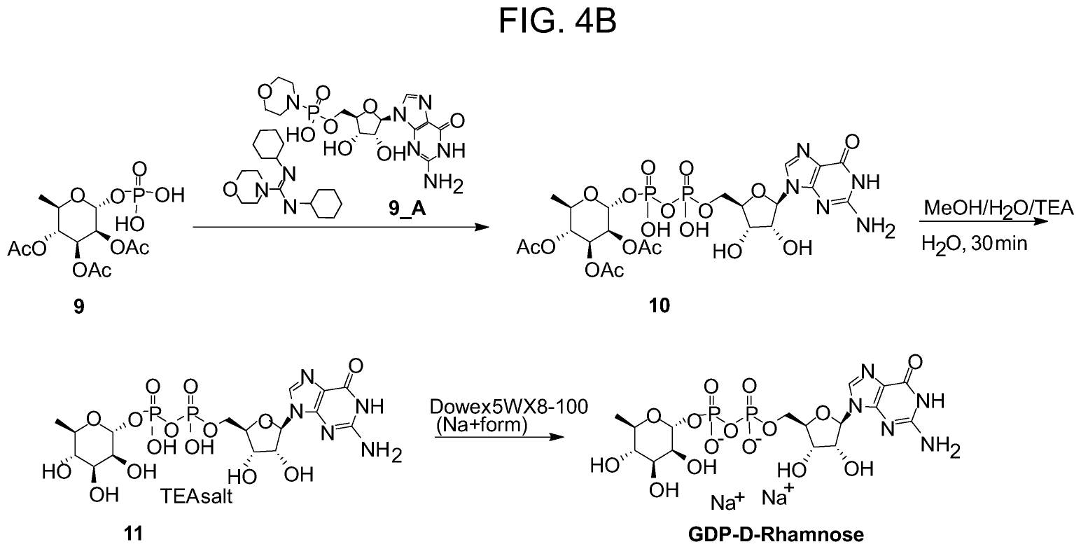

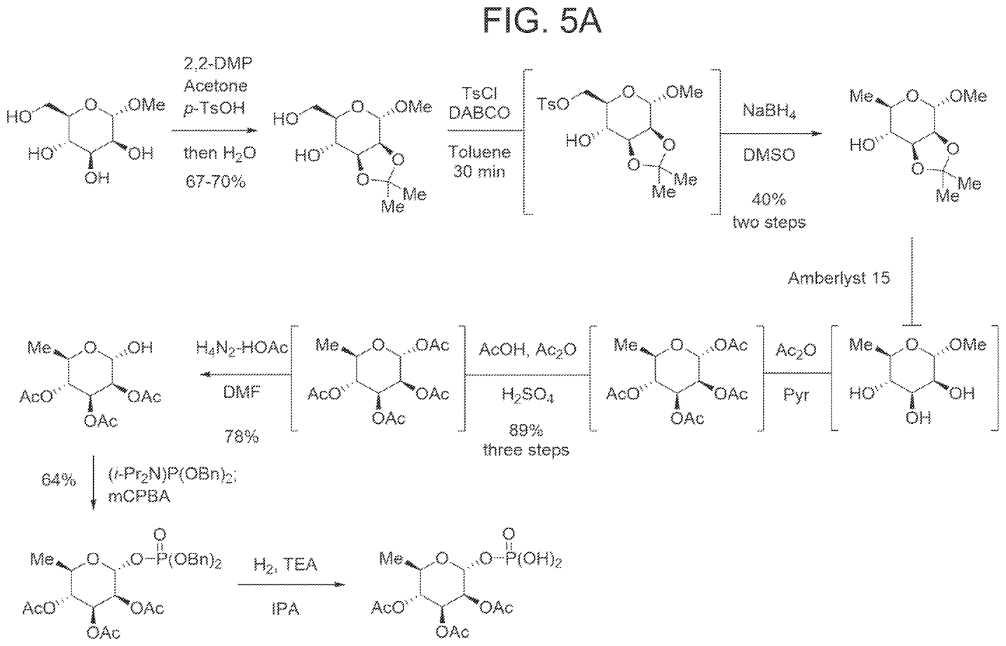

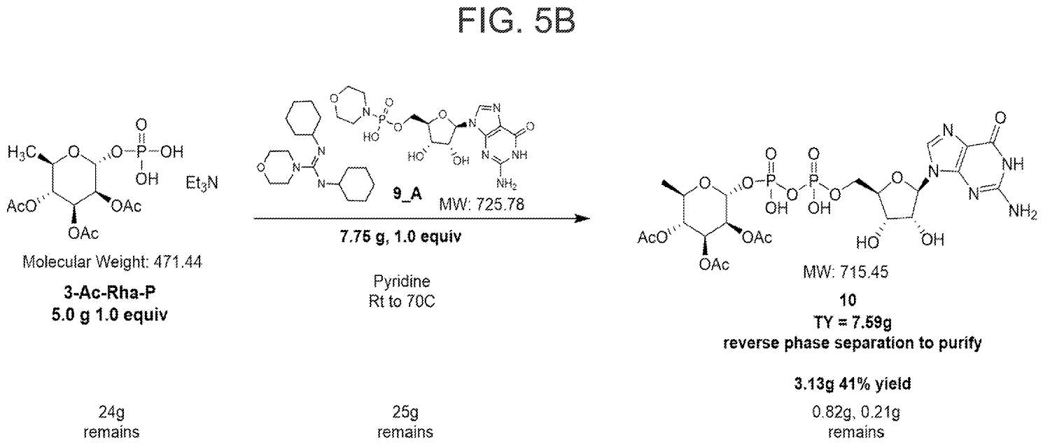

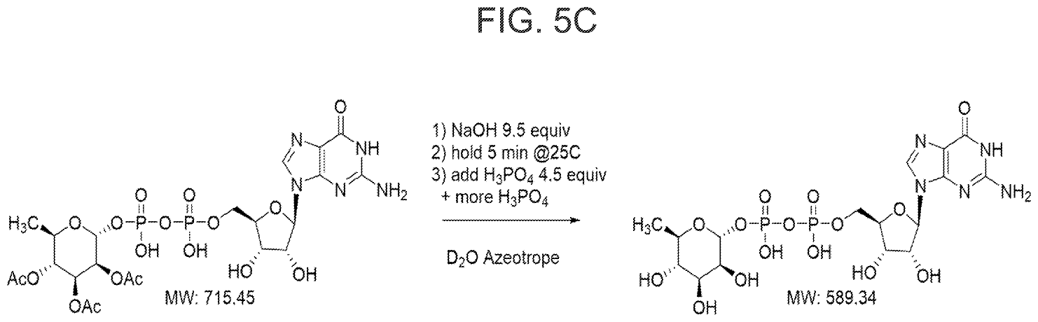

In one aspect, the present invention provides rhamnose-derived compounds, such as GDP-D-rhamnose and derivatives thereof, that inhibit fucosylation of proteins produced mammalian cell culture. Without intending to be limited by theory, such compounds may act as inhibitors of GDP-mannose-4,6-dehydratase (GMD). Exemplary compounds of the present invention include GDP-D-rhamnose (Formula I), Ac-GDP-D-rhamnose (Formula II), and sodium rhamnose phosphate (Formula IIII), structures of which are provided at FIG. 1. An exemplary method of synthesis of compounds of the present invention is provided at FIGS. 4A and 4B (for Ac-GDP-D-rhamnose) and FIGS. 4A, 4B and 4C (GDP-D-rhamnose) and discussed in greater detail at Example 1. A second exemplary method of synthesis of compounds of the present invention is provided at FIGS. 5A and 5B (for Ac-GDP-D-rhamnose) and FIGS. 5A, 5B and 5C (GDP-D-rhamnose).

The present invention also provides methods of producing proteins with reduced fucosylation, and hypofucosylated and nonfucosylated proteins, such as antibodies, by growing protein-producing cells in culture medium comprising a fucosylation inhibitor of the present invention, such as GDP-D-rhamnose, Ac-GDP-D-rhamnose, and sodium rhamnose phosphate, for examples, at concentrations of 6 mM or higher, or 10 mM or higher.

The present invention also provides proteins, such as antibodies, made by methods of the present invention, and methods of treatment of diseases, e.g. cancer, with these proteins (e.g. antibodies).

Because nonfucosylated antibodies exhibit greatly enhanced ADCC compared with fucosylated antibodies, antibody preparations need not be completely free of fucosylated heavy chains to be therapeutically superior to fucosylated antibodies. Residual levels of fucosylated heavy chains will not significantly interfere with the ADCC activity of a preparation substantially of nonfucosylated heavy chains. Antibodies produced in conventional CHO cells, which are fully competent to add core fucose to N-glycans, may nevertheless comprise from a few percent up to 15% nonfucosylated antibodies. Nonfucosylated antibodies may exhibit ten-fold higher affinity for CD16, and up to 30- to 100-fold enhancement of ADCC activity, so even a small increase in the proportion of nonfucosylated antibodies may drastically increase the ADCC activity of a preparation. Any preparation comprising more nonfucosylated antibodies than would be produced in normal CHO cells in culture may exhibit some level of enhanced ADCC. Such antibody preparations are referred to herein as preparations having “reduced fucosylation.” Depending on the original level of nonfucosylation obtained from normal CHO cells, reduced fucosylation preparations may comprise as little as 40%, 30%, 20%, 10% and even 5% nonfucosylated antibodies. Reduced fucosylation is functionally defined as preparations exhibiting two-fold or greater enhancement of ADCC compared with antibodies prepared in normal CHO cells, and not with reference to any fixed percentage of nonfucosylated species.

In other embodiments the level of nonfucosylation is structurally defined. As used herein, nonfucosylated antibody preparations are antibody preparations comprising greater than 95% nonfucosylated antibody heavy chains, including 100%. Hypofucosylated antibody preparations are antibody preparations comprising less than or equal to 95% heavy chains lacking fucose, e.g. antibody preparations in which between 50 and 95% of heavy chains lack fucose, such as between 75 and 95%, and between 85 and 95%. Unless otherwise indicated, hypofucosylated refers to antibody preparations in which 50 to 95% of heavy chains lack fucose, nonfucosylated refers to antibody preparations in which over 95% of heavy chains lack fucose, and “hypofucosylated or nonfucosylated” refers to antibody preparations in which 50% or more of heavy chains lack fucose.

The level of fucosylation in an antibody preparation may be determined by any method known in the art, including but not limited to gel electrophoresis, liquid chromatography, and mass spectrometry. Unless otherwise indicated, for the purposes of the present invention, the level of fucosylation in an antibody preparation is determined by hydrophilic interaction chromatography (or hydrophilic interaction liquid chromatography, HILIC), essentially as described at Example 2. To determine the level of fucosylation of an antibody preparation, samples are denatured treated with PNGase F to cleave N-linked glycans, which are then analyzed for fucose content. LC/MS of full-length antibody chains is an alternative method to detect the level of fucosylation of an antibody preparation, but mass spectroscopy is inherently less quantitative.

### Therapeutic Uses and Methods of the Invention

In some embodiments, such as treatment of cancer or infection, it may be desired to deplete immunosuppressive cells, such as regulatory T cells (Tregs), to allow a more robust anti-tumor or anti-infective immune response, or to deplete the tumor infected cells themselves. In such cases antibodies (or antigen binding fragments thereof) raised against cell surface proteins that are preferentially or exclusively expressed on the immunosuppressive cells, or against cell surface proteins that are preferentially or exclusively expressed on the tumor cells (e.g. tumor antigens) or infected cells themselves, are produced in mammalian cell lines grown in the presence of rhamnose-related fucosylation inhibitors of the present invention to produce populations of hypofucosylated or nonfucosylated antibodies with enhanced ADCC activity. In other cases, in which pathological inflammation causes disease, such as autoimmune disorders, hypofucosylated or nonfucosylated antibodies produced in mammalian cell lines grown in the presence of rhamnose-related fucosylation inhibitors of the present invention are specific for cell surface proteins that are preferentially or exclusively expressed on the inflammatory cells themselves.

In preferred embodiments of the present therapeutic methods, the subject is a human.

Examples of cancers that may be treated using hypofucosylated or nonfucosylated antibodies produced by the methods of the present invention include bone cancer, pancreatic cancer, skin cancer, cancer of the head or neck, breast cancer, lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, a hematological malignancy, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, metastatic cancers, and any combinations of said cancers. In preferred embodiments, the cancer is selected from MEL, RCC, squamous NSCLC, non-squamous NSCLC, CRC, CRPC, squamous cell carcinoma of the head and neck, and carcinomas of the esophagus, ovary, gastrointestinal tract and breast. The present methods are also applicable to treatment of metastatic cancers.

Other cancers include hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, and any combinations of said cancers.

The present invention is further illustrated by the following examples, which should not be construed as limiting. The contents of all figures and all references, patents and published patent applications cited throughout this application are expressly incorporated herein by reference.

### EXAMPLE 1

### Exemplary Synthesis of Fucosylation Inhibitors

The exemplary synthetic method for making fucosylation inhibitors of the present invention provided at FIGS. 4A and 5B is discussed here in greater detail.

Step 1.

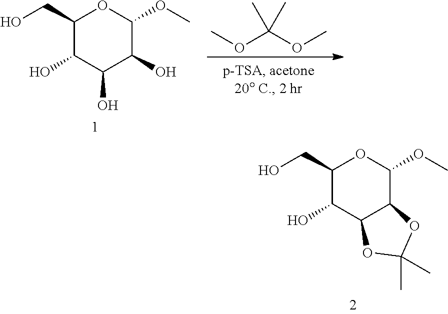

To a solution of compound 1 (150 g, 772 mmol, 1 eq), 2,2-DIMETHOXYPROPANE (402 g, 3.86 mol, 473 mL, 5 eq) and PTSA (6.65 g, 38.6 mmol, 0.05 eq) in acetone (750 mL) was stirred for 2 h at 20° C. TLC (ethyl acetate, SM (R_{f})=0.01, Product (R_{f})=0.38) showed the reaction was complete. The mixture was added water (150 mL). After 30 min, PTSA was neutralized with 5% aq NaHCO₃. Acetone was removed in vacuo and the aqueous phase was washed with petroleum ether to take apart the diisopropylidene then with DCM (3*200 mL). The organic layer was dried (Na₂SO₄) and concentrated in vacuo to give compound 2 (100 g, 55%) as off-white solid used into the next step without further purification.

Step 2.

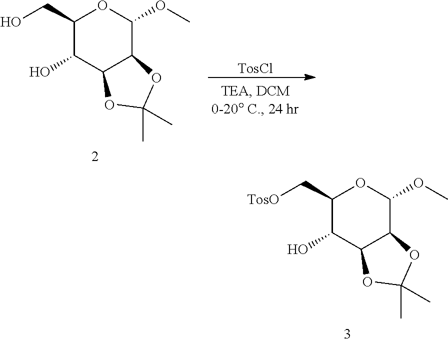

To a solution of compound 2 (100 g, 426 mmol, 1 eq) in DCM (700 mL) was added TEA (56.1 g, 554 mmol, 77.25 mL, 1.3 eq) and TosCl (105 g, 554 mmol, 1.3 eq). The mixture was stirred at 20° C. for 16 h.

TLC (Petroleum ether: Ethyl acetate=1:1, Product (R_{f})=0.43) indicated compound 2 was consumed completely. CH₂Cl₂ (200 mL) was added, and the solution was successively washed with saturated NaHCO₃ (5×300mL) and H₂O (3×300 mL), dried (MgSO₄), and evaporated to a syrup. The residue was purified by column chromatography (SiO₂, Petroleum ether/Ethyl acetate=5/1 to 2/1) to give compound 3 (100 g, 60% yield) was obtained as a light yellow oil.

Step 3.

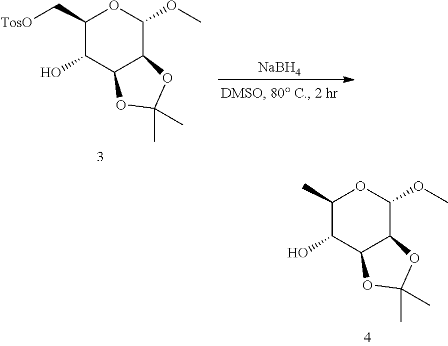

Carry out the two reactions in parallel.
To a solution of compound 3 (45.0 g, 115 mmol, 1 eq) in DMSO (450 mL) under N2 was cooled to 20° C. and NaBH4 (21.9 g, 579 mmol, 5 eq) was slowly added with stirring. The mixture was stirred at 80° C. for 2 h. TLC (Petroleum ether: Ethyl acetate=2:1, Product (R_{f})=0.43) indicated compound 3 was consumed completely. Two reactions were combined here. The mixture was quenched with ice H₂O (1400 mL), The mixture was stirred for 15 min, and then washed with EtOAc (1000 mL), dried (Na₂SO₄), and evaporated. The residue was purified by column chromatography (SiO₂, Petroleum ether/Ethyl acetate=5/1 to 2/1) to give compound 4 (40 g, 79% yield) as a light yellow oil.

Step 4.

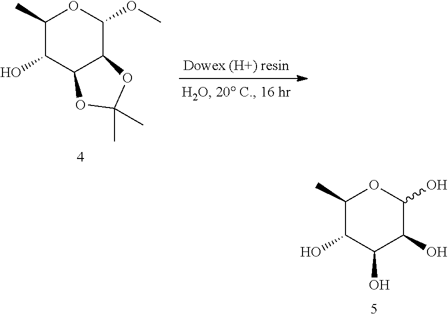

To a solution of compound 4 (40 g, 183 mmol, 1 eq) in H₂O (2000 mL) was added Dowex 50 H+ resin (300 g). The mixture was stirred at 80° C. for 24 hr. TLC (Dichloromethane: Methanol=3:1, Product (R_{f})=0.15) indicated compound 4 was consumed completely. The reaction mixture was filtered and concentrated under reduced pressure to give compound 5 (30 g, crude) was obtained as a light yellow oil.

Step 5.

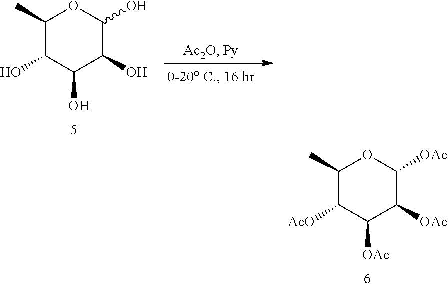 [SMILES: C[C@H]1OC(O)[C@@H](O)[C@@H](O)[C@@H]1O ; CC(=O)O[C@H]1O[C@H](C)[C@@H](OC(C)=O)[C@H](OC(C)=O)[C@@H]1OC(C)=O]

To a solution of compound 5 (30.0 g, 182 mmol, 1 eq) in Py (300 mL) was added DMAP (4.47 g, 36.5 mmol, 0.2 eq) and Ac₂O (149 g, 1.46 mol, 136 mL, 8 eq), The mixture was stirred at 20° C. for 12 hr. TLC (Petroleum ether: Ethyl acetate=3:1, Product (R_{f})=0.43) indicated compound 5 was consumed completely. The reaction mixture was quenched by addition H₂O (300 mL), and then diluted with EtOAC (500 mL). The organic layer was washed with 1N HCl (300 mL×2), dried over Na₂SO₄, filtered and concentrated under reduced pressure to give a residue. The residue was purified by column chromatography (SiO₂, Petroleum ether/Ethyl acetate=10/1 to 5:1) to give compound 6 (30 g, 49% yield) was obtained as a light yellow oil.

Step 6.

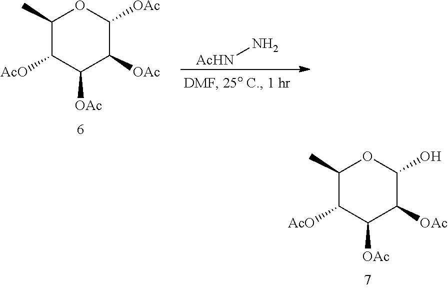 [SMILES: CC(=O)O[C@H]1O[C@H](C)[C@@H](OC(C)=O)[C@H](OC(C)=O)[C@@H]1OC(C)=O ; CC(=O)NN ; CC(=O)O[C@H]1[C@H](OC(C)=O)[C@@H](C)O[C@H](O)[C@H]1OC(C)=O]

The compound 6 (20.0 g, 60.1 mmol, 1 eq) is dissolved in DMF (110 mL). Acetic acid;hydrazine (8.31 g, 90.2 mmol, 1.5 eq) is added and the mixture is stirred at 25° C. under N₂ for 3 h. TLC (Petroleum ether: Ethyl acetate=1:1, Product (R_{f})=0.24) indicated compound 6 was consumed completely. The reaction mixture was quenched by addition H₂O 300 mL at 0° C., and then extracted with EtOAc (200 mL×2). The combined organic layers were washed with brine (100 mL×3), dried over Na₂SO₄, filtered and concentrated under reduced pressure to give a residue. Compound 7 (12.0 g, crude) was obtained as light yellow oil was used into the next step without further purification.

Step 7.

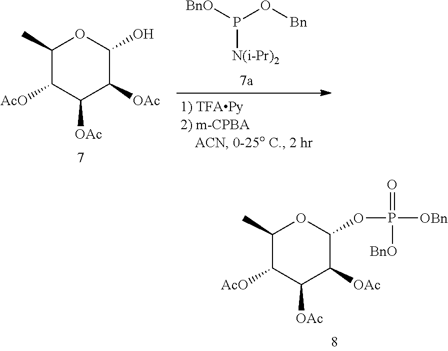

The compound 7 (12.0 g, 41.3 mmol, 1 eq) was co-evaporated with —30mL ACN twice and then 50 mL of ACN was added. The 7a (15.7 g, 45.4 mmol, 15 mL, 1.1 eq) in 40 mL of ACN was added. The mixture was cooled to 0° C. TFA.Py (1 M, 74 mL, 1.8 eq) was added dropwise at 0-5° C. The mixture was stirred at 25° C. for 1 hr. Cooled to 0° C. and m-CPBA (15.1 g, 74.4 mmol, 85% purity, 1.8 eq) in 40 mL of ACN was added dropwise at 0° C. The mixture was stirred at 25° C. for 1 hr. TLC (Petroleum ether: Ethyl acetate=2:1, Product (R_{f})=0.24) indicated compound 7 was consumed completely. Sat. Na₂SO₃(400 mL) and EtOAc(600 mL) was added and the mixture was stirred at 25° C. for 20 min. The organic phase was separated and washed with Sat. Na₂SO₃(300 mL*2) and brine(300 mL), dried over Na₂SO₄, filtered. The residue was purified by column chromatography (SiO₂, Petroleum ether/Ethyl acetate=2/1) to give compound 8 (10.0 g, 43% yield) was obtained as a light yellow oil.

Step 8.

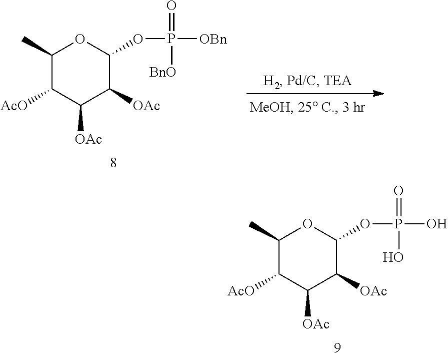 [SMILES: CC(=O)O[C@@H]1[C@H](OC(C)=O)[C@@H](OP(=O)(OCc2ccccc2)OCc2ccccc2)O[C@H](C)[C@H]1OC(C)=O ; CC(=O)O[C@@H]1[C@H](OC(C)=O)[C@@H](OP(=O)(O)O)O[C@H](C)[C@H]1OC(C)=O]

To a solution of compound 8 (4.00 g, 7.27 mmol, 1 eq) in MeOH (200 mL) was added Pd/C (10%, 4.0 g), 3.63 mL TEA under N₂ atmosphere. The suspension was degassed and purged with H2 for 3 times. The mixture was stirred under H₂ (30 Psi) at 25° C. for 3 hr. TLC (Petroleum ether: Ethyl acetate=1:1, Product (R_{f})=0.05) indicated compound 8 was consumed completely. The mixture was filtered through celite, the filter cake was washed with MeOH (30 mL) and concentrated under reduced pressure to give compound 9 (1.5 g, 55.7% yield) was obtained as a light yellow oil.

Step 9.

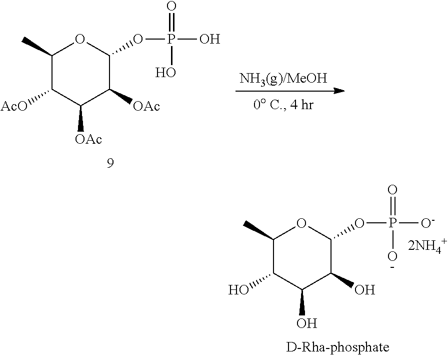 [SMILES: CC(=O)O[C@@H]1[C@H](OC(C)=O)[C@@H](OP(=O)(O)O)O[C@H](C)[C@H]1OC(C)=O ; C[C@H]1O[C@H](OP(=O)([O-])O)[C@@H](O)[C@@H](O)[C@@H]1O]

To a solution of compound 8 (1.5 g, 4.05 mmol, 1 eq) was added NH₃/MeOH (7 M, 70 mL, 120 eq). The mixture was stirred at 25° C. for 12 h. LCMS(et14769-65-pla, Rt=0.235 min) showed desired MS was detected. The reaction mixture was filtered and filter cake concentrated under reduced pressure to give a residue. Lyophilization the product. Compound D-Rha-phosphate (0.6 g, 61% yield) was obtained as light yellow oil.

Step 10.

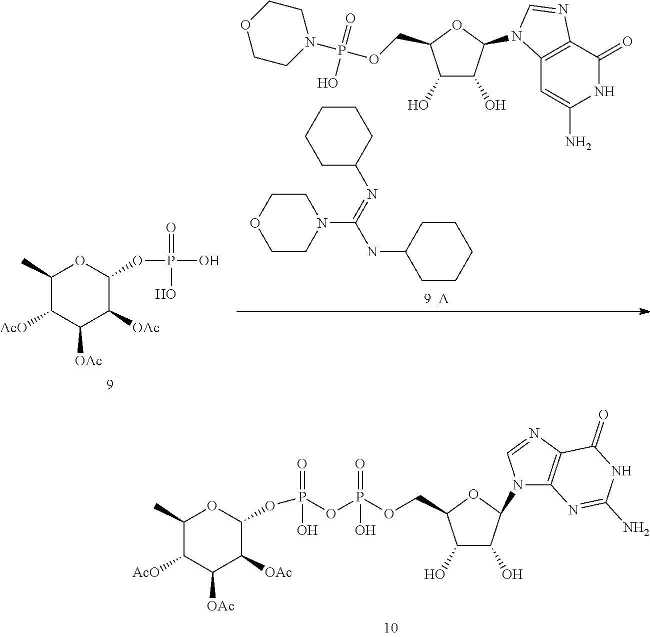

The compound 9 (0.1 g, 270 umol, 1 eq) was co-evaporated with Py (1 mL×2). The compound 9_A (98.0 mg, 135 umol, 0.5 eq) was added and the mixture was co-evaporated with Py (1 mL×2). Tetrazole (0.45 M, 1.20 mL, 2 eq) was added and the mixture was co-evaporated with Py (1 mL×2). Py (2 mL) was added and degassed with N₂. The mixture was stirred at 25° C. for 40 h. LCMS(et14769-78-p1D, Rt=1.157 min) showed Reactant 1 was remained. Several new peaks were shown on LC-MS and desired compound was detected. The reaction mixture was concentrated under reduced pressure to give a residue. The residue was purified by prep-HPLC (neutral condition). Lyophilization to give the mixture compound 10 and compound 9 (20 mg, 61% yield) was obtained as light yellow oil.

Step 11.

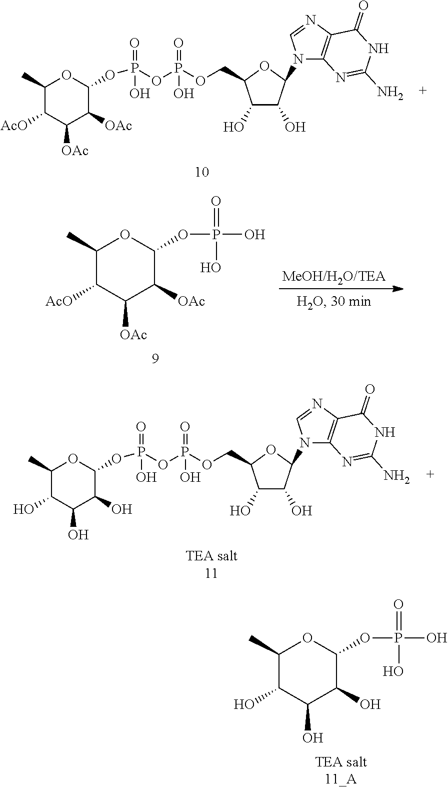 [SMILES: CC(=O)O[C@@H]1[C@H](OC(C)=O)[C@@H](OP(=O)(O)OP(=O)(O)OC[C@H]2O[C@@H](n3cnc4c(=O)[nH]c(N)nc43)[C@H](O)[C@@H]2O)O[C@H](C)[C@H]1OC(C)=O ; CC(=O)O[C@@H]1[C@H](OC(C)=O)[C@@H](OP(=O)(O)O)O[C@H](C)[C@H]1OC(C)=O ; C[C@H]1O[C@H](OP(=O)(O)OP(=O)(O)OC[C@H]2O[C@@H](n3cnc4c(=O)[nH]c(N)nc43)[C@H](O)[C@@H]2O)[C@@H](O)[C@@H](O)[C@@H]1O ; C[C@H]1O[C@H](OP(=O)(O)O)[C@@H](O)[C@@H](O)[C@@H]1O]

The mixture of compound 10 and compound 9 (20 mg) was dissolved in H₂O (0.5 mL). A solution of MeOH/H20/TEA (0.5 mL) was added. The mixture was stirred at 30° C. for 20 min. LCMS(et14769-83-pla) showed desired ms was detected. The resulting mixture was added H₂O (6 mL) and lyophilization 3 times. The mixture compound 11 and compound 11_A (20 mg) was obtained as light yellow oil.

Step 12.

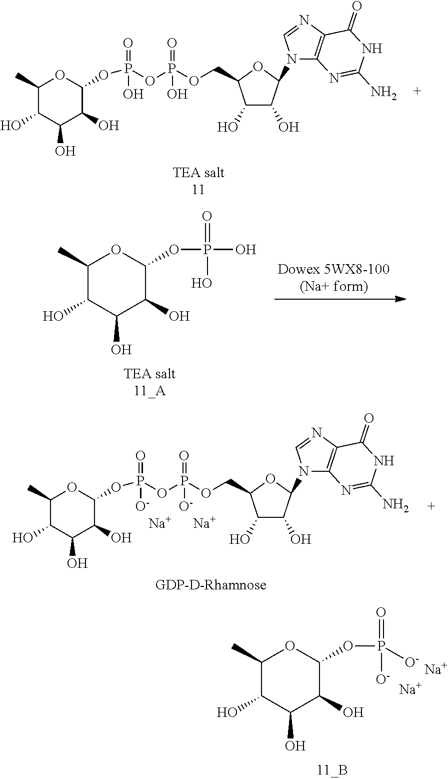

The mixture of compound 11 and compound 11_A (20 mg) was eluated through Dowex 5WX8-100 (Na⁺ form) with non-ion H₂O (300 mL). The elution was lyophilized. The mixture compound GDP-D-Rhamnose and compound 11_B (15 mg) was obtained as light yellow solid.

### EXAMPLE 2

### Assay to Determine Percentage Nonfucosylated Antibodies in a Sample

Nonfucosylated antibody preparations may be analyzed to determine the percentage of afucosylated heavy chains essentially as follows.

Antibodies are first denatured using urea and then reduced using DTT (dithiothreitol). Samples are then digested overnight at 37° C. with PNGase F to remove N-linked glycans. Released glycans are collected, filtered, dried, and derivatization with 2-aminobenzoic acid (2-AA) or 2-aminobenzamide (2-AB). The resulting labeled glycans are then resolved on a HILIC column and the eluted fractions are quantified by fluorescence and dried. The fractions are then treated with exoglycosidases, such as α(1-2,3,4,6) fucosidase (BKF), which releases core α(1,6)-linked fucose residues. Untreated samples and BKF-treated samples are then analyzed by liquid chromatography. Glycans comprising α(1,6)-linked fucose residues exhibit altered elution after BKF treatment, whereas nonfucosylated glycans are unchanged. The oligosaccharide composition is also confirmed by mass spectrometry. See, e.g., Zhu et al. (2014) MAbs 6:1474.

Percent nonfucosylation is calculated as one hundred times the molar ratio of (glycans lacking a fucose α1,6-linked to the first GlcNac residue at the N-linked glycan at N297 of the antibody heavy chain) to (the total of all glycans at that location, including both glycans lacking fucose and those having α1,6-linked fucose).

TABLE 7
Summary of the Sequence Listing

| SEQ ID NO. | Description |
|---|---|
| 1 | hamster GMD protein (NP_001233625.1) |
| 2 | hamster GMD DNA (NM_001246696.1) |
| 3 | human GMD protein (NP_001491.1) |
| 4 | human GMD DNA (NM_001500.4) |

### Equivalents:

Those skilled in the art will recognize, or be able to ascertain using no more than routine experimentation, many equivalents of the specific embodiments disclosed herein. Such equivalents are intended to be encompassed by the following claims.

1. A method of producing a protein with reduced fucosylation from a mammalian cell line expressing the protein, said method comprising:
a. culturing the mammalian cell line in culture medium comprising a compound comprising rhamnose; and
b. isolating the protein with reduced fucosylation.

2. The method of claim 1 wherein the isolated protein with reduced fucosylation comprises at least 20% nonfucosylated protein.

3. The method of claim 2 wherein the isolated protein with reduced fucosylation comprises at least 40% nonfucosylated protein.

4. The method of claim 3 wherein the isolated protein with reduced fucosylation is hypofucosylated or nonfucosylated.

5. The method of claim 1 wherein the compound is GDP-D-rhamnose, Ac-GDP-D-rhamnose, or sodium rhamnose phosphate.

6. The method of claim 5 wherein the compound is Ac-GDP-D-rhamnose.

7. The method of claim 5 wherein the compound is GDP-D-rhamnose.

8. The method of claim 5 wherein the compound is present in the culture medium at 6 mM or more.

9. The method of claim 8 wherein the compound is present in the culture medium at 10 mM or more.

10. The method of claim 5 wherein the compound is present in the culture medium during substantially all of the time that the mammalian cell line produces the protein with reduced fucosylation.

11. The method of claim 1 wherein the protein is an antibody.

12. The method of claim 11 wherein the isolated antibody with reduced fucosylation exhibits two-fold or greater enhancement of ADCC compared with the same antibody produced in the same cell line in the absence of fucosylation inhibitor, as determined by the method described in Example 2.

13. A protein with reduced fucosylation made by the method of claim 1.

14. An antibody with reduced fucosylation made by the method of

11.

15. A method treating cancer comprising administering a protein of claim 13 to a patient in need thereof.

16. A method treating cancer comprising administering an antibody of claim 14 to a patient in need thereof.

17. Ac-GDP-D-rhamnose.

18. D-rhamnose phosphate.


SEQUENCE LISTING (4 sequences)

SEQ ID NO: 1 - protein, 372 aa, Cricetulus griseus
MAHAPASCPSSRNSGDGDKGKPRKVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAIKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFMHIGKTIVWEGKNENEVGRCKETGKIHVTVDLKYYRPTEVDFLQGDCSKAQQKLNWKPRVAFDELVREMVQADVELMRTNPNA

SEQ ID NO: 2 - DNA, 1606 bp, Cricetulus griseus
agactgtggcggccgctgcagctccgtgaggcgactggcgcgcgcacccacgtctctgtcggcccgctgccggttccacggttccactcctccttccactcggctgcacgctcacccgcccgcggcgacatggctcacgctcccgctagctgcccgagctccaggaactctggggacggcgataagggcaagcccaggaaggtggcgctcatcacgggcatcaccggccaggatggctcatacttggcagaattcctgctggagaaaggatacgaggttcatggaattgtacggcgatccagttcatttaatacaggtcgaattgaacatttatataagaatccacaggctcatattgaaggaaacatgaagttgcactatggtgacctcaccgacagcacctgcctagtaaaaatcatcaatgaagtcaaacctacagagatctacaatcttggtgcccagagccatgtcaagatttcctttgacttagcagagtacactgcagatgttgatggagttggcaccttgcggcttctggatgcaattaagacttgtggccttataaattctgtgaagttctaccaggcctcaactagtgaactgtatggaaaagtgcaagaaataccccagaaagagaccacccctttctatccaaggtcgccctatggagcagccaaactttatgcctattggattgtagtgaactttcgagaggcttataatctctttgcggtgaacggcattctcttcaatcatgagagtcctagaagaggagctaattttgttactcgaaaaattagccggtcagtagctaagatttaccttggacaactggaatgtttcagtttgggaaatctggacgccaaacgagactggggccatgccaaggactatgtcgaggctatgtggctgatgttacaaaatgatgaaccagaggactttgtcatagctactggggaagttcatagtgtccgtgaatttgttgagaaatcattcatgcacattggaaagaccattgtgtgggaaggaaagaatgaaaatgaagtgggcagatgtaaagagaccggcaaaattcatgtgactgtggatctgaaatactaccgaccaactgaagtggacttcctgcagggagactgctccaaggcgcagcagaaactgaactggaagccccgcgttgcctttgacgagctggtgagggagatggtgcaagccgatgtggagctcatgagaaccaaccccaacgcctgagcacctctacaaaaaattcgcgagacatggactatggtgcagagccagccaaccagagtccagccactcctgagaccatcgaccataaaccctcgactgcctgtgtcgtccccacagctaagagctgggccacaggtttgtgggcaccaggacggggacactccagagctaaggccacttcgcttttgtcaaaggctcctctgaatgattttgggaaatcaagaagtttaaaatcacatactcattttacttgaaattatgtcactagacaacttaaatttttgagtcttgagattgtttttctcttttcttattaaatgatctttctatgaaccagcaaaaaaaaaaaaaaaaaaaa

SEQ ID NO: 3 - protein, 372 aa, Homo sapiens
MAHAPARCPSARGSGDGEMGKPRNVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAVKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFLHIGKTIVWEGKNENEVGRCKETGKVHVTVDLKYYRPTEVDFLQGDCTKAKQKLNWKPRVAFDELVREMVHADVELMRTNPNA

SEQ ID NO: 4 - DNA, 1665 bp, Homo sapiens
ctccctgcacggcctcccgtgcgcccctgtcagactgtggcggccggtcgcgcggtgcgctctccctccctgcccgcagcctggagaggcgcttcgtgctgcacacccccgcgttcctgccggcaccgcgcctgccctctgccgcgctccgccctgccgccgaccgcacgcccgccgcgggacatggcacacgcaccggcacgctgccccagcgcccggggctccggggacggcgagatgggcaagcccaggaacgtggcgctcatcaccggtatcacaggccaggatggttcctacctggctgagttcctgctggagaaaggctatgaggtccatggaattgtacggcggtccagttcatttaatacgggtcgaattgagcatctgtataagaatccccaggctcacattgaaggaaacatgaagttgcactatggcgatctcactgacagtacctgccttgtgaagatcattaatgaagtaaagcccacagagatctacaaccttggagcccagagccacgtcaaaatttcctttgacctcgctgagtacactgcggacgttgacggagttggcactctacgacttctagatgcagttaagacttgtggccttatcaactctgtgaagttctaccaagcctcaacaagtgaactttatgggaaagtgcaggaaataccccagaaggagaccacccctttctatccccggtcaccctatggggcagcaaaactctatgcctattggattgtggtgaacttccgtgaggcgtataatctctttgcagtgaacggcattctcttcaatcatgagagtcccagaagaggagctaatttcgttactcgaaaaattagccggtcagtagctaagatttaccttggacaactggaatgtttcagtttgggaaatctggatgccaaacgagattggggccatgccaaggactatgtggaggctatgtggttgatgttgcagaatgatgagccggaggacttcgttatagctactggggaggtccatagtgtccgggaatttgtcgagaaatcattcttgcacattggaaaaaccattgtgtgggaaggaaagaatgaaaatgaagtgggcagatgtaaagagaccggcaaagttcacgtgactgtggatctcaagtactaccggccaactgaagtggactttctgcagggcgactgcaccaaagcgaaacagaagctgaactggaagccccgggtcgctttcgatgagctggtgagggagatggtgcacgccgacgtggagctcatgaggacaaaccccaatgcctgagcagcgcctcggagcccggcccgccctccggctacaatccccgcagagtctccggtgcagacgcgctgcggggatggggagcggcgtgccaatctgcgggtcccctgcggcccctgctgccgctgcgctgtcccggccgcaagagcggggccgccccgccgaggtttgtagcagccgggatgtgaccctccagggtttgggtcgctttgcgtttgtcgaagcctcctctgaatggctttgtgaaatcaagatgttttaatcacattcactttacttgaaattatgttgttacacaacaaattgtggggccttcaaattgtttttctcttttcatattaaaaatggtctttctgtgaactagca